(19) United States
(12) Patent Application Publication
(10) Pub. No.: US 2025/0147207 A1
(43) Pub. Date: May 8, 2025

(54) DIRECTING ELECTROMAGNETIC RADIATION

(71) Applicant: 12180235 Canada Ltd., CentrePort (CA)

(72) Inventors: David Allan Prystupa, Pinawa (CA); John Stephen Pacak, Winnipeg (CA)

(21) Appl. No.: 18/938,847

(22) Filed: Nov. 6, 2024

Related U.S. Application Data
(60) Provisional application No. 63/596,842, filed on Nov. 7, 2023.

(51) Int. Cl. G02B 5/08; A61L 2/08; A61L 2/10; G02F 1/17

(52) U.S. Cl. CPC G02B 5/0891; A61L 2/085; A61L 2/10; G02F 1/172

(57) ABSTRACT

A method for regulating flow of electromagnetic radiation into a volume such as a duct includes defining on a reflective surface an array of two or more ridges which are arranged side by side and projecting toward the source. Each ridge is formed by reflective ridge surfaces converging toward an apex so that the reflection directions of the surfaces are different. The ridges are arranged relative to the source so that first reflective ridge surface reflects primarily toward the upstream location and second reflective ridge surface reflects onto the first reflective ridge surface of a next adjacent ridge and thereby primarily toward the upstream location so that the array of ridges acts to form a restriction to flow of radiation toward the downstream location. This can increase an amount of the radiation within the duct by reflecting the radiation back into the duct and to reduce an amount of radiation escaping the duct.

This invention relates generally to a method for regulating flow of electromagnetic radiation using reflective surfaces. The methods described herein may be used to increase the electromagnetic radiation energy density in a region proximate to the surface of a duct, wall or corridor. The methods described herein may be used to direct diffuse electromagnetic radiation in a selected direction.

### RELATED APPLICATIONS

This disclosure is related to Apparatus for Reflecting an Incident Ray of Electromagnetic Radiation; U.S. application Ser. No. 17/378,144 filed Jul. 16 2021 and published as US 2022/0016278 and corresponding PCT application PCT/CA2021/050976 published as WO2022/011472 published 20 Jan. 2022 hereafter referred to as the CHAMBER patents. The disclosure of this publication is hereby incorporated herein by reference.

This disclosure is related to Reflective Surface for a Photochemistry Chamber, PCT application PCT/CA2023/050038 filed Jan. 13, 2023 which claims priority from U.S. provisional application 63/299,535 filed Jan. 14, 2022 and published as WO2023/133643 on 20 Jul. 2023 by the present inventors hereafter referred to as the WALL patent. The disclosure of this publication is hereby incorporated herein by reference.

### BACKGROUND INFORMATION

The invention relates generally to the fields of photochemistry chambers, illumination, and thermal management for electromagnetic radiation in UV, visible and infrared ranges, respectively. The general problem to be solved is to regulate the flux of electromagnetic radiation within a selected volume proximate to one or more surfaces defining a selected volume while minimizing the intrusion of optical elements into the selected volume.

Advantages of the embodiments described herein include minimizing optical material requirements, minimizing the volume required for an optical device, and allowing the unimpeded flow of a material through the selected volume.

Common proposals known in the art include lining surfaces at least partially bounding the selected volume with isotropic reflectors. These solutions suffer from five main drawbacks. Firstly, radiation diffusely escapes from the selected volume in any unbounded direction (generally the direction of material flow). The flux lost is proportional to the solid angle of the unbounded directions. Secondly, the average path length between reflections (and energy loss via absorption) is less than optimal. Thirdly, the reflectivity of a material varies with angle of incidence and since distribution of incident angles is broad, at least some of the reflections are at angles of incidence corresponding to lower reflectivity (and more absorption loss). Fourth, commercially available isotropic reflectors have high cost. Fifth, commercially available isotropic reflectors based on halogen containing polymers may release toxic fumes when heated above 300 C.

In the above CHAMBER patents, the second problem is solved by causing a radiant flux is made to reflect back and forth between highly reflective smooth curved surfaces within a confined or partially confined volume. Highly reflective dielectric mirrors are used to minimize absorption losses at the surfaces and can be optimized for specific angles of incidence solving the third problem. Dielectric mirrors are comprised of fully oxidized minerals with very high melting points and do not emit toxic fumes. The surfaces are arranged to maximize the average path length between reflections thereby reducing absorption losses. This gives a higher radiation flux in the selected volume than isotropic reflectors with the same reflectivity. In the above CHAMBER patents, the first problem may be solved completely by using directional radiation sources without altering the direction of material flow. For isotropic radiation sources the first problem may be solved at the cost of directing material flow around obstacles. The CHAMBER patents provide an optimal solution for cases where there are no constraints to the shape of the surfaces. However, there are many circumstances in which the shape of the surfaces is constrained by walls separated by a constant distance or there is a flow requirement that is not consistent with optimally curved surfaces described in the CHAMBER patent. For example, the selected volume may lie within a pre-existing duct or corridor with a fixed rectangular or circular cross section perpendicular to axial flow direction of the selected volume.

The present invention may provide one or more of the following features:

A first objective of the present disclosure is to provide a method for reducing the flux of electromagnetic radiation escaping from a selected volume of a duct or corridor in a generally axial direction subject to the constraint that perturbation to the cross-sectional profile of the duct or corridor is minimized.

A second objective of the present disclosure is to provide a method that increases the flux of electromagnetic radiation within a selected volume of a duct or corridor which can be applied to pre-existing infrastructure.

A third objective of the present disclosure is to provide a method that increases the flux of electromagnetic radiation within a selected volume of a duct or corridor which gives a cost saving over prior art methods.

A fourth objective of the present disclosure is to provide a method that increases the flux of electromagnetic radiation within a selected volume of a duct or corridor which is fire safe.

A fifth objective of the present disclosure is to provide a method to adaptively regulate radiative energy transport from a first region of space to a second region of space.

A sixth objective of the present disclosure is to provide a method to direct a beam of electromagnetic radiation from a diffuse source in a selected direction.

### SUMMARY OF THE INVENTION

According to a first aspect of the invention there is provided a method for regulating flow of electromagnetic radiation comprising
providing a source of electromagnetic radiation;
arranging the source to emit electromagnetic radiation in a direction into a volume,
placing a reflective surface in the volume to receive the radiation thereon;
defining on the reflective surface an array of two or more ridges which are arranged side by side in a first direction across the reflective surface;
wherein each ridge projects forwardly from a base toward the source;
wherein each ridge has a first reflective ridge surface and a second reflective ridge surface where the first and second surfaces extend longitudinally in a second direction transverse to the first direction;
wherein the first reflective ridge surface and the second reflective ridge surface converge toward an apex extending longitudinally along the second direction;
wherein the first reflective ridge surface and the second reflective ridge surface are arranged so that at least some of the radiation impinges on at least one of the first reflective ridge surface and the second reflective ridge surface;
wherein the first reflective ridge surface and the second reflective ridge surface are arranged such that the radiation incident on the first reflective ridge surface is primarily reflected in a first reflection direction and such that the radiation incident on the second reflective ridge surface is primarily reflected in a second reflection direction different from that of the first reflection direction.

The invention as disclosed herein may provide one or more of the following features:

A first objective of the present disclosure is to provide a method for reducing the flux of electromagnetic radiation escaping from a selected volume of a duct or corridor in a generally axial direction subject to the constraint that perturbation to the cross-sectional profile of the duct or corridor is minimized.

A second objective of the present disclosure is to provide a method that increases the flux of electromagnetic radiation within a selected volume of a duct or corridor which can be applied to pre-existing infrastructure.

A third objective of the present disclosure is to provide a method that increases the flux of electromagnetic radiation within a selected volume of a duct or corridor which gives a cost saving over prior art methods.

A fourth objective of the present disclosure is to provide a method that increases the flux of electromagnetic radiation within a selected volume of a duct or corridor which is fire safe.

A fifth objective of the present disclosure is to provide a method to adaptively regulate radiative energy transport from a first region of space to a second region of space.

A sixth objective of the present disclosure is to provide a method to direct a beam of electromagnetic radiation from a diffuse source in a selected direction.

According to a second aspect of the invention there is provided a duct section extending along at least a part of the length of a duct for transporting air or other fluid comprising
at least one duct wall of the duct section;
a source of electromagnetic radiation located within the duct arranged to emit electromagnetic radiation at least partly in a direction toward the wall;
the radiation being arranged to cause sterilization of the air in the duct;
a reflective surface on the wall to receive the radiation incident thereon;
the reflective surface including a series of at least two ridges which are arranged side by side in a first direction across the reflective surface;
wherein each ridge projects forwardly from the wall toward the source;
wherein each ridge has a first reflective ridge surface and a second reflective ridge surface where the first and second surfaces extend longitudinally in a second direction transverse to the first direction;
wherein the first reflective ridge surface and the second reflective ridge surface converge toward an apex extending longitudinally along the second direction;
wherein the first reflective ridge surface and the second reflective ridge surface are arranged so that at least some of the radiation impinges on at least one of the first reflective ridge surface and the second reflective ridge surface;
wherein the first reflective ridge surface and the second reflective ridge surface are arranged such that the radiation incident on the first reflective ridge surface is primarily reflected in a first reflection direction and such that the radiation incident on the second reflective ridge surface is primarily reflected in a second reflection direction different from that of the first reflection direction;
the first and second reflective ridge surfaces being arranged to increase an amount of the radiation within the duct section by reflecting the radiation back into the duct section and to reduce an amount of radiation escaping the duct section.

According to a third aspect of the invention there is provided an apparatus for restricting flow of radiation in a direction from an upstream location to a downstream location comprising
a source of electromagnetic radiation located arranged to emit electromagnetic radiation,
a reflective surface arranged to at least part of the radiation incident thereon;
the reflective surface including a series of at least two ridges which are arranged side by side in a first direction across the reflective surface;
wherein each ridge projects forwardly from the wall toward the source;
wherein each ridge has a first reflective ridge surface and a second reflective ridge surface where the first and second surfaces extend longitudinally in a second direction transverse to the direction;
wherein the first reflective ridge surface and the second reflective ridge surface converge toward an apex extending along the direction;
wherein the first reflective ridge surface and the second reflective ridge surface are arranged so that at least some of the radiation impinges on at least one of the first reflective ridge surface and the second reflective ridge surface;
wherein the first reflective ridge surface and the second reflective ridge surface are arranged such that the radiation incident on the first reflective ridge surface is primarily reflected in a first reflection direction and such that the radiation incident on the second reflective ridge surface is primarily reflected in a second reflection direction different from that of the first reflection direction;
where the ridges are arranged relative to the source so that first reflective ridge surface reflects primarily toward the upstream location and second reflective ridge surface reflects onto the first reflective ridge surface of a next adjacent ridge and thereby primarily toward the upstream location so that the array of ridges acts to form a restriction to flow of radiation toward the downstream location.

According to a fourth aspect of the invention there is provided an apparatus for directing radiation from a source in a required direction comprising
a black body source of electromagnetic radiation arranged to emit radiation partly in the required direction,
a reflective shroud at the source for guiding the radiation emitted from the source which is not in the required direction;
the reflective shroud comprising:
   one or more reflective surfaces at least partly surrounding the source at locations different from the required direction and arranged to receive at least part of the radiation incident thereon;
   the or each reflective surface including a series of at least two ridges which are arranged side by side in a first direction across the reflective surface;
   wherein each ridge projects forwardly from the wall toward the source;
   wherein each ridge has a first reflective ridge surface and a second reflective ridge surface where the first and second surfaces extend longitudinally in a second direction transverse to the first direction;
   wherein the first reflective ridge surface and the second reflective ridge surface converge toward an apex extending longitudinally along the second direction;
   wherein the first reflective ridge surface and the second reflective ridge surface are arranged so that at least some of the radiation impinges on at least one of the first reflective ridge surface and the second reflective ridge surface;
wherein the first reflective ridge surface and the second reflective ridge surface are arranged such that the radiation incident on the first reflective ridge surface is primarily reflected in a first reflection direction and such that the radiation incident on the second reflective ridge surface is primarily reflected in a second reflection direction different from that of the first reflection direction;
the or each reflective surface being arranged such that a portion on one side of the source generally opposes a portion on an opposite side;
the first and second reflective ridge surfaces being arranged such that the radiation incident on the first and second ridge surfaces of each portion is reflected toward the source, toward the opposed portion, or toward the required direction;
wherein radiation directed toward the source is absorbed and re-emitted.

There are four outcomes in order of likelihood (i) reflection toward the source, absorption at the source and re-emission in a different direction; (ii) reflection toward an opposed side where (i) or (iii) may occur; (iii) reflection in the required direction (iv) absorption and re-emission from the ridge surface. The arrangement recycles the energy of photons emitted in the wrong directions by returning the energy to the source for re-emission. A fraction of the re-emitted radiation is in the desired direction. This mechanism works for thermal radiation because all of the thermal energy is re-emitted as thermal photons. This mechanism is less effective for visible radiation because a large part of the visible photon energy absorbed by the source is converted to heat and re-emitted at longer wavelengths rather than the original visible wavelength.

Thus the disclosure relates to a low-profile optical valve that regulates the direction of electromagnetic radiation flux in a selected direction parallel to a base surface location wherein at least a portion of the radiation flux is directed toward the base surface location at an angle of incidence greater than zero degrees. The term “radiation flux” herein refers to the transport of energy by an ensemble of photons. The term “ray” herein refers to, and may be used interchangeably with the term “Poynting vector”, that is a vector in the direction of power flow of electromagnetic waves with magnitude specifying the power. A radiation flux may include a plurality of rays in different directions and magnitudes which summed give a net power flux. For brevity, the direction of radiation flux to be regulated is referred to as the “valve direction” herein and is specific to each location on the base surface. The valve direction is a unit vector parallel to the surface at the surface location pointing in the general direction of radiation flux from a radiation source. That is the dot product of the valve direction vector with the Poynting vector of the electromagnetic radiation to be regulated is always greater than zero. The optical valve operates to reduce the net flux of radiation in the valve direction by preferentially reflecting radiation flux in a direction with a vector component opposite to the valve direction.

In accordance with an important feature of the invention there is provided at least one base surface. The base surface is defined as the interface between a substrate material that is generally opaque to electromagnetic radiation and a medium material that transmits at least one design wavelength of electromagnetic radiation with low attenuation. The medium material may be vacuum, gas, liquid, or solid. The base surface may be stationary or in motion relative to a reference surface or a second base surface.

A combination of base surfaces may for example form the interface between a duct and air in a HVAC system, the air being transmissive to UV, visible and infrared wavelengths. The base surface may for example be the interface between an architectural structure such as a building wall and air in a corridor, room, or open area. The base surface may for example be the interface between a pipe material and a liquid such as water that transmits visible wavelengths and attenuates to varying degrees UV and infrared wavelengths. The base surface may for example be the interface between opaque cladding and the interior medium of a waveguide. The waveguide may be hollow (air or gas medium) or have an optically transmissive core comprised of an optical liquid, glass or plastic. The base surface may for example be integral to a satellite in orbit. The base surface may for example be integral to a conveyor belt.

In accordance with an important feature of the invention there is provided at least one electromagnetic radiation source. In some embodiments the radiation source may be proximate or integral to the base surface. In other embodiments the radiation source may be distant from the base surface. The radiation source may be a discrete source of electromagnetic radiation such as a black body radiator, bulb, discharge tube, filament or LED proximate or integral to the base surface. The discrete source of radiation may for example emit UV wavelengths for a photochemistry chamber defined at least in part by the base surface. The discrete source of radiation may for example emit visible wavelengths to illuminate a chamber defined at least in part by the base surface for human vision. Radiation emitted from the radiation source propagates in a plurality of directions with an angular distribution characteristic to the source of radiation. The radiation source may be the base surface itself together with adjacent fluid (air for example) that emits thermal radiation. Each region of the base surface and volume element of the fluid may have different temperatures and emit thermal radiation differently. The thermal radiation from each surface region or volume element may have a wavelength distribution that approximates a black body radiation distribution, modified by the emissivity of surface and fluid materials. The base surface may for example have high emissivity at wavelengths between 4000 nm and 25000 nm which, when combined with high reflectivity at wavelengths shorter than 4000 nm is known in the art to radiatively cool the base surface. The radiation source may for example be the sun irradiating a satellite in orbit. In this case the rays at any instant in time have a narrow range of incident angles relative to the base surface. However, the range of angles of incidence may be broader as a time average due to changes in orientation relative to the sun. The radiation may for example be rays from the sun Rayleigh scattered by the atmosphere.

In accordance with an important optional feature of the invention which can be used independently with any of the above or following features, a material flow is associated with and influenced by a base surface. The material flow may be air in a HVAC duct confined by one or more base surfaces. The material flow may be humans transiting a corridor or moving around a room or outdoor space bounded by at least one base surface. The material flow may be the flow of a fluid such as water in a pipe or channel. The material flow may be materials in an industrial process on a conveyor belt. In some embodiments, the material flow may be generally parallel to a base surface. In other embodiments the material flow may have a velocity component perpendicular to a base surface. The material flow may be influenced by the dimensions and texture of the base surfaces.

In accordance with an important feature of the invention there is provided a substrate material with a substrate surface that provides attachment points and structural support to the optical features described herein. In some embodiments, the substrate surface and base surface may coincide. In some embodiments, the substrate material is attached to the base surface and the substrate surface is displaced from the base surface by the substrate material thickness. The substrate surface may be functionally equivalent to the base surface for the purpose of confining the flow of a material. The substrate material thickness may be selected to keep the perturbations to flow (velocity, volume, turbulence) associated with a base surface below threshold values for each flow parameter. For example, in a HVAC duct 200 mm wide, a substrate thickness less than 1 mm will have minimal impact on air flow in the duct. For example, a substrate thickness of 100 mm may have minimal impact on air flow in an outdoor space such as a patio, but a thinner substrate may be preferred to reduce material cost.

In accordance with an important feature of the invention there is provided an array of two or more reflective ridges located on the substrate surface and oriented generally perpendicular to the local valve direction of the underlying base surface. The ridges are comprised of two or more joined ridge surfaces that have no point vertically displaced from the substrate surface by more than a height threshold value and extend in a direction perpendicular to the valve direction and perpendicular to the substrate surface normal by a distance greater than a length threshold value wherein two said ridge surfaces intersect the substrate. A first ridge surface intersecting the substrate surface has a surface normal component generally anti-parallel to the local valve direction and a second intersecting ridge surface intersecting the substrate surface has a surface normal component generally parallel to the local valve direction. In the case that the valve direction is constant, the intersections of ridge surfaces are straight lines and the surface normal components parallel to the substrate surface are exactly anti-parallel and parallel to the valve direction for the first and second ridge surfaces, respectively. In the case that the valve direction varies with location, the lines of intersection are curved and the projections of the ridge surface normal components parallel to substrate surface may deviate from exactly anti-parallel and parallel to the local valve direction. The height threshold value may scale with the size of the base surface. For a base surface with typical dimensions of 10 m or less appropriate threshold values are in the range of 0.1 mm to 10 mm. For a base surface with typical dimensions over 10 m, the threshold values may be scaled up proportionately. The length threshold value is a line integral along the geometric center of the ridge and scales proportionately to the shortest distance between the points of intersection of the first and second ridge surfaces, generally understood as the ridge width. The length threshold value is at least twice the ridge width.

In an important embodiment that may be used in combination with any of the preceding or following embodiments, the ridges have a generally triangular cross section wherein the center of a third side of the triangle is proximate or coincident with the substrate surface and has a normal anti-parallel to the normal of the substrate surface; a second side joined with the third side and inclined at an angle α to the first side that has a normal that resolved into components normal to the substrate surface (cos(α)) and anti-parallel to the valve direction (−sin(α)); and a second side joined with the third side and inclined at an angle β to the first side that has a normal resolved into components normal to the substrate surface (cos(β)) and parallel to the valve direction (sin(β)); wherein the first side and second side meet and are joined at an apex displaced from the substrate surface by no more than a height threshold value. The apex angle of the triangular ridge is hence γ=π/2−α−β.

In an important embodiment that may be used in combination with any of the preceding or following embodiments, the ridges may have an irregular polygonal cross section with one or more fourth sides inserted between the first and second sides described above for the triangular cross section wherein no point of the fourth sides is displaced from the substrate surface by more than the threshold value. For example, a nominally triangular ridge formed by folding or deforming a substrate material may have a generally rounded top at the apex angle that may be modeled as one or more fourth sides with slopes intermediate between the second and third sides. For example, the ridge may have a generally trapezoidal shape wherein a flat fourth side parallel to the substrate surface joins the first and second sides. The flat fourth side may for example reflect a different range of wavelengths than the first and second sides. A ridge of this type may function as a dichotic valve by preferentially passing one wavelength in the valve direction and preferentially reflecting a second wavelength anti-parallel to the valve direction.

In accordance with an important optional feature of the invention which can be used independently with any of the above or following features, successive ridges in an array of ridges are abutting: that is the second side of a first ridge intersects with and is joined with the first side of a second ridge proximate to or at the substrate surface. The term “proximate to the substrate surface” is understood herein to mean a point on a chord to a substrate surface with curvature in the valve direction. The term “at the substrate surface” is understood herein to mean a point on the substrate surface and is applicable if the substrate surface is a plane or if the substrate surface is a cylinder with axis in the valve direction.

In accordance with an important optional feature of the invention which can be used independently with any of the above or following features, the ridge array includes at least one substantially flat reflective surface proximate or integral with the substrate surface and parallel to the substrate surface joined to at least one first or second side of a ridge as described above. The flat reflective surface has reflectivity at a selected wavelength and design angle of incidence of at least 80%, preferably at least 90% and most preferably at least 95%.

In accordance with an important optional feature of the invention which can be used independently with any of the above or following features, the reflectivity of the ridge array at a design wavelength may change in response to an external stimulus wherein the external stimulus is a temperature or a voltage applied to the substrate layer. This feature may be used for example make the ridge array highly reflective at high ambient temperatures (to cool an architectural structure) and absorbing at low ambient temperatures (to absorb energy and heat an architectural structure). For example, the substrate layer may be comprised of a reflective layer, a liquid crystal layer, and a transparent layer which functions to retain the liquid crystal layer. In a first state, the liquid crystal layer transmits incident radiation to the reflective layer and transmits reflected radiation. In a second state, the liquid crystal layer absorbs incident radiation. A transition between the liquid crystal states may be temperature dependent. Alternately, the transition between liquid crystal states may be caused by applying a voltage across the liquid crystal layer. For example, the substrate layer may be comprised of a material that undergoes solid state phase transitions wherein the different states have different reflectivity at a design wavelength. Suitable materials are described for example by Rios et al in Adv. Mat. 2016, 28 4720-4726.

In accordance with an important optional feature of the invention which can be used independently with any of the above or following features, at least one ridge geometric parameter may be modified in response to an external stimulus wherein the external stimulus is one of electrical, mechanical, or thermal. The ridge geometric parameter may be an angle between ridge substrate surfaces, the height of a ridge, or the orientation of a ridge relative to the valve direction. The geometric parameter may for example be altered by an electrical stimulus applied to piezoelectric material attached to or integral to the substrate layer. The ridge array may for example be comprised of a micro mirror array and the geometric parameter is altered by mechanically changing the orientation of at least one mirror in the array (including with electrical activation). The geometric parameter may for example be altered by a change in temperature wherein at least a portion of a ridge is attached to or integral with a material that changes shape with a change in temperature. The material may for example be a shape memory polymer. The material may for example be a bimetallic strip. This feature may be used for example to configure the ridge array to either retard electromagnetic radiation flux in the valve direction or to pass electromagnetic radiation flux in the valve direction dependent on the ambient temperature. Retarding the radiation flux has the effect of increasing the radiation density and temperature proximate to the ridge array and passing radiation flux lowers the temperature via radiative cooling.

In an important embodiment that may be used in combination with any of the preceding or following embodiments, the substantially flat reflective surface is joined with the first side of a ridge and is located “upstream” of the first ridge in the array. That is the substantially flat reflective surface is displaced from the first ridge in the array in a direction opposite to the direction of radiation flux to be regulated (valve direction). The substantially flat reflective surface may for example be located immediately opposite a radiation source and reflect radiation from the source generally in a direction toward the radiation source. The substantially flat reflective surface may for example include a region displaced from the radiation source in the valve direction. In this case radiation incident on the flat reflective surface is reflected with a vector component in the valve direction. The first ridge in the array may for example be displaced from a radiation source in the valve direction such that radiation from the source strikes the leading surface of the first ridge (and subsequent ridges) at an acute angle and is reflected generally in a direction toward the radiation source.

In an important embodiment that may be used in combination with any of the preceding or following embodiments, a substantially flat reflective surface is located between the first and second sides of successive ridges. The flat reflective surface may for example be located in the shadow region of a first ridge relative to a specified radiation source. The flat reflective surface may for example function to modify the angular distribution of radiation reflected by the ridge array.

In an important embodiment that may be used in combination with any of the preceding or following embodiments, a substantially flat reflective surface is located within or proximate to a region with ridges following a curved pattern. That is the valve direction varies with location. Concentric ridges following a curved pattern may for example increase in density toward the center of the pattern and flat reflective regions are included in the pattern to keep the number of ridges per unit area approximately constant.

In accordance with an important optional feature of the invention which can be used independently with any of the above or following features, the substrate material may include pores which allow the passage of molecules through the substrate material between the medium material and the base surface. The pores may be an array of apertures. In some embodiments a masking reflective surface is positioned above and vertically displaced from the aperture so as to reflect radiation otherwise directly incident on the aperture. Molecules pass through a gap between the masking reflective surface and underlying ridge or substrate surface in a direction generally perpendicular to the aperture axis. In some embodiments the substrate material may be micro porous. Preferably the micro pore size is less than ¼ of the design wavelength of the system to minimize scattering effects.

The ridge period is defined as the center to center separation between adjacent ridges comprising the projected span onto the base surface of the first and second sides, optional fourth sides and an optional flat region. In an important embodiment that may be used separately or in combination with preceding or following embodiments, the ridge period is constant for a predefined number of ridges. Ray tracing simulations show that the optimal ridge parameters such as slope angles (α and β) found by optimizing a merit function vary slowly with ridge position relative to a radiation source. The merit function may for example be based on the radiation flux density in a volume upstream of the ridges: that is the merit function measures the flux reflected anti-parallel to the valve direction. From a fabrication cost perspective, it may be expedient to fabricate ridge sections containing a predefined number of ridges of constant period wherein the ridge period is selected to optimize the merit function of the set of ridges. The number of ridges may be selected by comparing the ridge merit function for ridge parameters optimized for each ridge individually with the merit function for ridge parameters optimized for a set of ridges and requiring that the difference is less than a predefined threshold.

In an important embodiment that may be used separately or in combination with preceding or following embodiments the ridge period is not constant. For example, the ridge period may increase with distance from a radiation source such that each successive ridge subtends the same solid angle from the radiation source. For example the ridge parameters may be individually optimized for each ridge according to a merit function as discussed above with the ridge period dependent on the parameters of each successive ridge.

In some embodiments the valve direction is constant over the width of a base surface and the ridges are linear perpendicular to the common valve direction.

In some embodiments a first part of a substrate surface has ridges with a constant first valve direction and a second part of a substrate surface has ridges with a second constant second valve direction.

In an important embodiment the first part of the substrate surface and second part of the substrate surface are separated by a third part of the substrate surface wherein ridges in substrate region and second substrate region have opposite valve directions. In this important embodiment, the optical valves aligned in opposite directions may for example function to confine radiation emitted proximate to the third part of the substrate surface to regions proximate to the third part of the substrate surface. The first, second and third parts of the substrate surface may have much higher reflectivity (and lower absorption losses) than regions external to the substrate regions. For example, the substrate regions may lie within a duct with a UV radiation source proximate to the third substrate region. The optical valves in the first and second substrate regions function to confine UV flux within the high reflectivity region allowing optical amplification of the UV dose as described in the CHAMBER patent by the current inventors.

In an important embodiment, an optical valve is positioned proximate to an electromagnetic radiation source and the optical valve functions to transmit electromagnetic radiation emitted within a first solid angle and to block electromagnetic radiation emitted into a second solid angle wherein at least a portion of the electromagnetic radiation emitted into the second solid angle is returned to the electromagnetic radiation source by the optical valve. That is the ridges of the optical valve operate to reflect radiation falling within a range of angles back to the radiation source. For example, the radiation source may be a planar thermal source emitting infrared radiation in all directions above the plane with Lambertian angular distribution. Infrared radiation emitted into the first solid angle emerges from the optical valve as a partially collimated beam. A portion of infrared radiation emitted into the second solid angle is returned to the thermal source where it is absorbed, thereby increasing the temperature and contributing to further thermal emission. The new photons are emitted in random directions with Lambertian angular distribution. A portion of the re-emitted radiation is emitted into the first solid angle thereby increasing the amplitude of radiation within the first solid angle. In the ideal case wherein the ridges are perfectly reflecting and the thermal source is a black body radiator, the cycle of emission into the second solid angle and re-emission into a random direction is repeated until the random direction falls within the first solid angle as required by conservation of energy. That is all of the energy supplied to the thermal radiator emerges as a partially collimated beam. This embodiment may be used for example as a broadband spectroscopic light source. This embodiment may be used for example to project infrared radiation in a desired direction for an industrial process such as soldering. This embodiment may be used for example to selectively provide radiant energy to regions of a building such as an arena containing people providing an energy saving over heating the building isotropically.

In some embodiments, the valve direction varies in a continuous fashion to form ridges with a curved shape. The ridges perpendicular to the local valve direction may for example follow a parabolic curve which, depending on the location of a radiation source may tend to focus or collimate radiation from the source. In some embodiments, the valve direction varies discontinuously. The ridges perpendicular to the local valve direction may for example form chevron shaped patterns.

In accordance with an important feature of the invention, the reflectivity at the designed wavelength of each reflective ridge surface of the incident radiation is at least 80% preferably at least 90% and most preferably at least 95%. Polished metallic surfaces and dielectric mirrors are suitable materials to produce the required reflectivity. In some embodiments wherein a transparent material with high refractive index abuts the ridge surface, total internal reflection occurs for angles of incidence above the critical angle and high reflectivity occurs for angles of incidence slightly below the critical angle. The design angle of incidence is chosen by measuring or modeling the angular distribution of radiation likely to be incident on each ridge surface and selecting a design angle of incidence at or near the intensity weighted average of the angular distribution of radiation. In some embodiments the selected high reflectivity wavelengths are the same for each ridge surface. In some embodiments the selected high reflectivity wavelengths may be different for each ridge surface.

In accordance with an important optional feature of the invention which can be used independently with any of the above or following features, the substrate surface and ridge surfaces are comprised of a dielectric material described in the above cited WALL patent by the current inventors. The WALL patent describes dielectric mirrors that are highly reflective at non-visible UV or infrared wavelengths and contain features that absorb or scatter visible light to form decorative patterns. The ridges described herein may be integral with a decorative pattern at visible wavelengths while being highly reflective at UV or infrared wavelengths.

In accordance with an important optional feature of the invention which can be used independently with any of the above or following features, the ridges are formed on a ridge tile which may be rigid. Each ridge tile provides a substrate surface with an array of ridges and may be attached to and overlay a fraction of the base surface. The ridge tiles may have different sizes and shapes that are assembled to conform to the shape of a base surface. The tiles may include a mark indicating the valve direction. The tiles may include code marks that specify the design wavelength, design angle of incidence, the number and geometry of ridges, the ridge height, and any decorative features as described in the above cited WALL patent by the current inventors.

In accordance with an important optional feature of the invention which can be used independently with any of the above or following features, there is provided a flexible ridge substrate. The flexible ridge substrate provides a substrate surface with an array of ridges that may be cut to the size of base surface and be attached to overlay the base surface or fraction thereof. The flexible ridge substrate may for example be comprised of a polymer film or thin glass sheet overlaid with a stack of dielectric layers fabricated to reflect radiation at a design wavelength and angle of incidence wherein the polymer film (and overlaid dielectric reflector) are formed to include an array of ridges of predetermined shape. The flexible ridge substrate may for example be comprised of a polymer film overlaid with a thin layer of metal such as Al, Au deposited on top to form a reflective surface at a design wavelength and angle of incidence wherein the polymer film (and overlaid metallic reflector) are formed to include an array of ridges of predetermined shape. The flexible ridge substrate may optionally include an adhesive layer for attachment to the base surface. The flexible substrate may include code marks that specify the design wavelength, design angle of incidence, the number and geometry of ridges, the ridge height, and any decorative features as described in the above cited WALL patent.

The ridge substrate may be rigid or flexible. It will be appreciated that dielectric mirrors are often formed as deposited layers on a support. Thus the reflective material may define the whole of the structure without any supporting substrate, the reflective material may be applied in a thin layer which is attached to a surface of the substrate or the reflective material may be directly deposited onto the substrate.

In an important embodiment that may be used in combination with any of the above or following embodiments, the base substrate is an outdoor wall that bounds a region of space on one side and arrays of ridges are added to reflect infrared radiation anti-parallel to a valve direction so as to retain thermal radiation and increase the temperature within a region proximate to the wall.

In an important embodiment that may be used in combination with any of the preceding or following embodiments, the base substrate is an outdoor wall that bounds a region of space on one side and arrays of ridges are added to reflect infrared radiation incident in the valve direction so as to reduce thermal radiation and lower the temperature within a region proximate to the wall past the ridges in the valve direction. That is the ridges operate to shade a region in the valve direction.

In an important embodiment that may be used in combination with any of the preceding or following embodiments, the base substrate is an outdoor wall that bounds a region of space on one side and arrays of ridges are added to reflect infrared radiation anti-parallel to a valve direction so as to retain thermal radiation and increase the temperature within a region proximate to the wall when the ambient temperature is below a first threshold temperature and the ridges are configured (change of ridge angles or reflectivity) so as to reduce thermal radiation and lower the temperature within a region proximate to the wall when the ambient temperature is above a second threshold temperature.

In an important embodiment that may be used in combination with any of the preceding or following embodiments, the base substrate is a corridor that joins a higher temperature part of a building and a lower temperature part of a building wherein arrays of ridges are added to the corridor walls to reflect infrared radiation so as to reduce the radiative transfer of energy from the higher temperature part of the building to the lower the temperature part of the building. The corridor may for example join an ice rink at a lower temperature a spectator area at a higher temperature.

In an important embodiment that may be used in combination with any of the preceding or following embodiments, the base surface is integral to the walls of a heated chamber, for example a muffle furnace or an oven. For example, arrays of ridges are added at the periphery of the oven chamber to reflect infrared radiation toward the interior of the oven chamber. The arrays of ridges may be fabricated on substrates of materials such as glass, ceramic, tungsten, silicon carbide, or other material that retains its form at high temperatures.

In an important embodiment that may be used in combination with any of the preceding or following embodiments, the base surface is a HVAC duct and UV sterilizing radiation at a wavelength between 180 nm and 300 nm is emitted into the duct. In one example, a section of the HVAC duct proximate to the radiation source is covered with ridge tiles as described above with the valve direction of the ridge tiles directed away from the radiation source on each side of the radiation source. The ridge tiles may be dimensioned to match the dimensions of the HVAC duct. In a second example a section of the HVAC duct proximate to the radiation source is covered with flexible ridge material as described above with the valve direction of the ridge tiles directed away from the radiation source on each side of the radiation source. Preferably the UV wavelength is about 265 nm. Preferably the ridge tiles contain about 20 ridges. Preferably the ridge height is about 0.5 mm. Preferably the ridge angle α is between 35 degrees and 55 degrees. Preferably the ridge angle β is between 45 degrees and 65 degrees.

In accordance with an important optional feature of the invention which can be used independently with any of the above or following features, the ridge array substrate and material underlying the base surface are flexible. The ridge array (and underlying layers) may for example be extended across a deployment surface in a first state and folded or rolled into a storage volume in a second state wherein the selected state depends upon a condition of the deployment surface or adjacent regions. The condition may for example be a temperature or a flux of radiation. In cold climate regions, the ridge array may for example be deployed on a deployment surface in the first state during winter months to confine radiant flux inside a building and stored in the second state during summer months. The ridge array may for example be deployed as a blind in the first state across a window opening to reflect thermal radiation away from the window at night and stored in the second state during daylight. In hot climate regions, the season and time of day may be reversed.

In accordance with an important optional feature of the invention which can be used independently with any of the above or following features, the ridge array is reflective at a first range of wavelengths and transmissive at a second range of wavelengths wherein the underlying substrate layer is also transmissive at the second range of wavelengths. The base surface and underlying material may be either transmissive or absorbing at the second range of wavelengths. For example, the ridge array may include a surface layer comprised of a multi-layer dielectric stack that is transmissive at visible wavelengths in the range 400 to 650 nm and reflective at infrared wavelengths greater than 1000 nm. In some embodiments, the substrate surface is parallel to the ridge surface and radiation in the first range of wavelengths is reflected and substantially all radiation in the second range of wavelengths is transmitted in substantially the same direction as the incident radiation suffering only a small lateral displacement. A small fraction of radiation incident at the apex of a ridge or at a valley between ridges may be transmitted with a change in direction in this case giving the visual appearance of a line. In an alternate embodiment, the interior of a ridge is comprised of a solid material that is transmissive in the second range of wavelengths. In this embodiment, each ridge functions as a small prism and the transmitted radiation is refracted.

In accordance with an important optional feature of the invention which can be used independently with any of the above or following features, an optically flat transparent thin film overlays the ridge array. The optically flat thin film has parallel surfaces such that radiation incident on a first surface exits the second surface in the same direction as the incident radiation. The optically transparent thin film functions to prevent particulate matter such as dust entering the spaces between ridges. Secondly, the optically transparent thin film may trap pockets of air, aerogel, or another transparent material that reduce the thermal conductivity from the thin film to the base surface.

In accordance with an important optional feature of the invention which can be used independently with any of the above or following features, the substrate layer includes a layer of thermal insulation.

In some embodiments ridge arrays are arranged to illuminate a sample volume for a machine vision system. The ridge arrays are positioned generally to the side of the sample region and operate to direct radiation emitted at the top of the sample region toward the side and bottom of the sample.

### BRIEF DESCRIPTION OF THE DRAWINGS

FIG. 1 shows a schematic longitudinal cross-sectional view along a first embodiment of the invention in which a duct for air includes two reflective ridge arrays on opposing sides of the duct cooperating with a source.

FIG. 2 is a cross sectional view along the lines 2-2 of FIG. 1 and shows the reflective ridge arrays on each wall of the duct.

FIG. 3 is an enlarged view of one portion of the duct of FIG. 1 showing the path or rays emitted by the source as they impact on the ridge surfaces.

FIG. 4 is an enlarged cross-sectional view of the reflective ridge array showing the layers making up the array and attached to a wall of the duct.

FIG. 5 shows a schematic longitudinal cross-sectional view along a second embodiment of the invention in which the reflective ridge arrays operate as an optical valve.

FIG. 6 shows a schematic longitudinal cross-sectional view along a third embodiment of the invention includes a black body radiator with two reflective ridge arrays on opposing sides of a shroud confining the radiation.

FIG. 7A shows a schematic top view of an array of parallel ridges.

FIG. 7B shows a schematic top view of an array of straight ridge segments.

FIG. 7C shows a schematic top view of an array of curved ridge segments.

FIG. 7D shows a schematic top view of an isotropic scattering surface according to prior art.

FIG. 8 shows the relationship between dose and material length for a reflective ridge array and for a flat surface in a duct.

FIG. 9 shows the relationship between dose and ridge height.

FIG. 10 shows the relationship between dose and reflectivity for ridge arrays in a duct.

FIG. 11 shows the amplitude of emission as a function of angle for a smooth bore black body radiator and a black body radiator with ridge arrays.

### DETAILED DESCRIPTION

Embodiments of the present disclosure are described in detail with reference to the accompanying drawings. Detailed descriptions of constructions or processes known in the art may be omitted to avoid obscuring the subject matter of the present disclosure. Further in the following description of the present disclosure, various specific definitions found in the following description are provided to give a general understanding of the present disclosure, and it is apparent to those skilled in the art that the present disclosure can be implemented without such definitions.

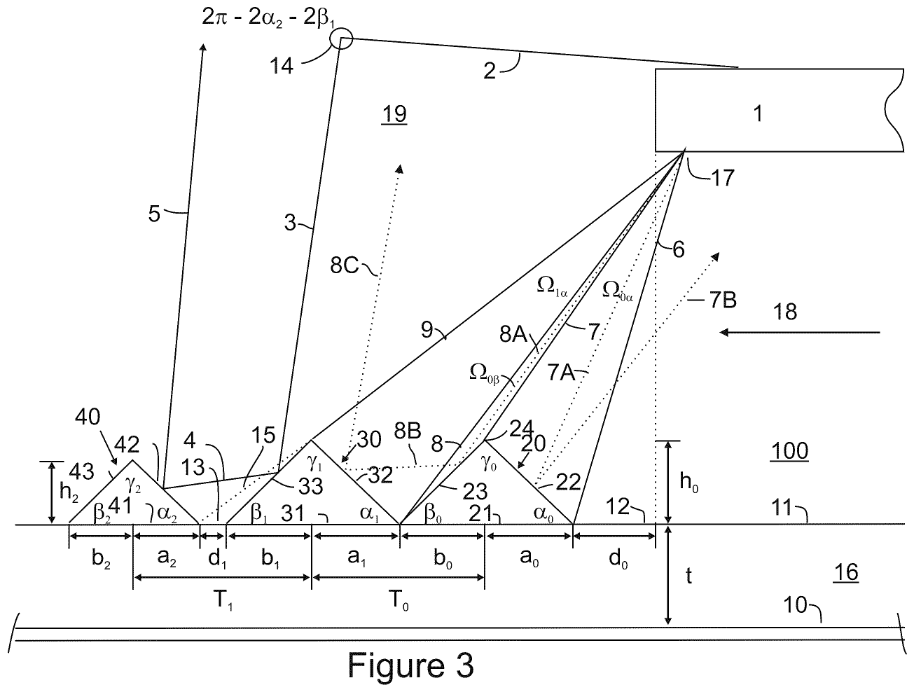

FIG. 3 shows a schematic view of the ridge arrangement of the invention generally indicated at 100. A substrate material 16 with thickness t is attached to and overlies a base surface 10 typically defined by a support wall. However the material 16 may in some arrangements have sufficient structural strength to define the mechanical support. Smooth and reflective substrate surface 11 is displaced from the base surface 11 by the thickness t. Preferably the RMS surface irregularity is less than ¼ of a design wavelength of electromagnetic radiation. More preferably the RMS surface irregularity is less than 1/20 of a design wavelength of electromagnetic radiation. The substrate surface 11 preferably has reflectivity of at least 80% at the design wavelength for a design angle of incidence. More preferably the substrate surface 11 has reflectivity of at least 90% at the design wavelength for a design angle of incidence. Most preferably the substrate surface 11 has reflectivity of at least 95% at the design wavelength for a design angle of incidence. In some embodiments the thickness t may be zero in which case the substrate surface and base surfaces coincide. In some embodiments the base surface and substrate surface are nearly parallel. In some embodiments (not shown) the substrate surface is inclined at an angle to the base surface.

The substrate surface 11 and features thereon are bounded by optical medium 19 which is weakly absorbing or transparent at the design wavelength. The optical medium may for example be a mixture of gasses such as air in a HVAC conduit, indoor space or outdoor space. The optical medium may for example be helium or nitrogen in a cryogenic chamber. The optical medium may for example be an inert gas such as argon or nitrogen in a reaction chamber. The optical medium may for example be gas at low density in space or a vacuum chamber. The optical medium may for example be a liquid such as water in a water treatment plant, pool, or pipe. The optical medium may for example be a solution in a flow through reaction chamber. The optical medium may for example be a solid such as glass or plastic in an optical conduit.

The substrate surface 11 and features thereon is illuminated by electromagnetic radiation source 1 which emits at least some electromagnetic radiation directly or indirectly toward substrate surface 11. The source of electromagnetic radiation may for example be a bulb, filament, glowbar, discharge tube, LED or any electronic device that emits electromagnetic radiation. The source of electromagnetic radiation may for example be the sun. The electromagnetic radiation received by substrate surface 11 and features thereon is calculated by integrating emitted radiation from all points 17 on the radiation source (or equivalently rays passing through a virtual surface enclosing the radiation source). Ray of electromagnetic radiation 2 from source 1 may be incident on a particle 14 and suffer scattering in a different direction toward substrate surface 11 as ray 3. The particle 14 may for example be a gas molecule Rayleigh scattering radiation. The particle 14 may for example be a dust or pollen particle.

The substrate surface 11 has a plurality of ridge features shown schematically at 20, 30 and 40 thereon. The ridges are generally triangular so that a first ridge 20 closest to radiation source 1 has first base 21 along substrate surface 11, a first surface 22 inclined at angle do to substrate surface 11 and having normal component anti-parallel to a direction 18 along the duct typically parallel to the substrate surface 11, and second surface 23 having normal component parallel to the direction 18 and inclined at angle β₀ to substrate surface 11. Sides 22 and 23 meet at apex 24 with apex angle γ₀ with vertical displacement h₀ from substrate surface 11. The projection of side 22 onto the substrate surface 11 is a₀ and the projection of side 23 onto substrate surface 11 is b₀. For abutting ridges with the same dimensions, the ridge period is T=a₀+b₀. Preferably h₀ is between 0.1 mm and 10 mm. Due to limits of fabrication methods, apex 24 may be slightly rounded with the rounded edge represented by one or more of fourth surfaces (not shown) with angles of inclination intermediate between sides 22 and 23. Side 22 may be displaced by a distance do from a projection of radiation source 1 onto substrate surface 11. For example, radiation source 1 may be a discharge tube oriented along the axis of a HVAC duct and do is an axial displacement from the edge of the discharge tube to the first ridge 20 in a sequence of two or more ridges.

Rays 6 and 7 from point 17 on radiation source 1 are incident at the edges of side 22 and define a solid angle Ω_{0α} of radiation incident on side 22. Note that all solid angles noted on FIG. 3 include angles encompassing the ridge length in planes perpendicular to the schematic drawing. Each point 17 on radiation source 1 radiates into a different solid angle with a different intensity. By integrating over all parts of radiation source 1, the intensity at side 22 as a function of angle of incidence may be calculated and a design angle of incidence is selected to maximize reflected intensity. The optimization may be for side 22 individually or for a plurality of ridge sides. Specifically, the material for the surface of side 22 is selected to maximize reflectivity at the design angle of incidence for a design wavelength. The reflective surface may for example be a dielectric mirror as described hereinbefore. Optimal values of d₀ may be found by modeling the propagation of radiation from all regions of the source to all surfaces within a defined test volume and calculating the radiation flux within the test volume for each value of do and selecting the value corresponding with the highest radiation flux.

Rays 7 and 8 from point 17 on radiation source 1 are incident at the edges of side 23 and define a solid angle of radiation incident on side 23. The average angle of incidence is near grazing for side 23 and the reflective material on side 23 is hence selected for high reflectivity at grazing angles. The material choice is broad as most materials with smooth surfaces are highly reflective at grazing angles. The solid angle Ω_{0β} for side 23 is smaller than the solid angle Ω_{0α} for side 21 with the difference increasing with increasing ridge height. Drawn to scale, the difference would be less than shown.

Second ridge 30 has a base 31 along substrate surface 11, first surface or side 32 having normal component anti-parallel to valve direction 18 inclined at angle α₁ to substrate surface 11, and second side 33 having normal component parallel to valve direction 18 and inclined at angle β₁ to substrate surface 11. Sides 32 and 33 meet with apex angle γ₁ with vertical displacement h₀ from substrate surface 11. The projection of side 32 onto substrate surface 11 is a₁ and the projection of side 33 onto substrate surface 11 is b₁. The period between peaks of the first and second ridges is T₀=a₁+b₀. Rays 8 and 9 from point 17 on radiation source 1 define solid angle Ω_{1α} for side 32 which is smaller than solid angle Ω_{0α} for side 22 (by 3 degrees as shown, but the difference would be smaller if drawn to scale). In general total benefit obtained by the ridge arrangement of the invention scales with the sum of solid angles subtended by the ridges in the array or ridges. The solid angle subtended by a ridge decreases with distance from the radiation source. Beyond a certain number of ridges the incremental benefit of adding an additional ridge exceeds the cost. The cost and benefit functions are application specific.

Ray 9 is shown extrapolated beyond ridge surface 32 at with a dashed line at 15 illustrating that ridge surface 33 is in the shadow of ridge surface 32: that is there is no direct path from radiation source 1 to ridge surface 33. In this circumstance, a flat region of length d1 indicated at 13 may be inserted into the array of ridges such that the next ridge slope 42 is directly illuminated by source 1. The shadow region including ridge surface 33 and flat region 13 are indirectly illuminated by radiation source 1 and may consequently receive less electromagnetic radiation than directly illuminated ridge surface 32. In some embodiments surfaces in the shadow region may include pores (not shown) that allow passage of gasses such as water vapor between optical medium 19 and substrate material 16. The pores may be an array of apertures. The pores may be included in a micro porous substrate material.

Third ridge 40 has first side 41 along substrate surface 11, second side 42 having normal component anti-parallel to valve direction 18 inclined at angle α₂ to substrate surface 11, and third side 43 having normal component parallel to valve direction 18 and inclined at angle β₂ to substrate surface 11. Sides 42 and 43 meet with apex angle with vertical displacement h₂ from substrate surface 11 illustrating that the height of ridges in a sequence may be different. The projection of side 42 onto substrate surface 11 is a₂ and the projection of side 43 onto substrate surface 11 is b₂. The period between peaks of the second and third ridges is T₁=a₂+d₁+b₁.

Ray 3 scattered by particle 14 toward surface 33 of ridge 30 is reflected by surface 33 toward surface 42 of ridge 40 as shown as reflected ray 4. Ray 4 is reflected by surface 42 in the direction indicated by reflected ray 5. This double bounce ray suffers energy loss at surfaces 33 and 42 whereas rays incident on the leading ridge surface such as ray 6 on surface 22 suffer energy loss at only one surface. Hence it is advantageous to select ridge angles such that the leading surfaces specified by the alpha angles subtend a larger solid angle and receive a greater proportion of incident radiation than the trailing surfaces specified by the beta angles. In practice, this means that the alpha angles are less than or equal to the beta angles in general. The angle between incident ray 3 and reflected ray 5 between ridges with triangular cross section can be shown to be 2π−2β₂−2β₁ in general. When α₂+β₁=π/2 rays 3 and 5 are anti-parallel. However, the anti-parallel case is not optimal for applications with a discrete radiation source because the reflected ray 5 would be directed back into the radiation source and absorbed. It is advantageous instead to choose ridge angles such that the reflected rays are not incident on the radiation source.

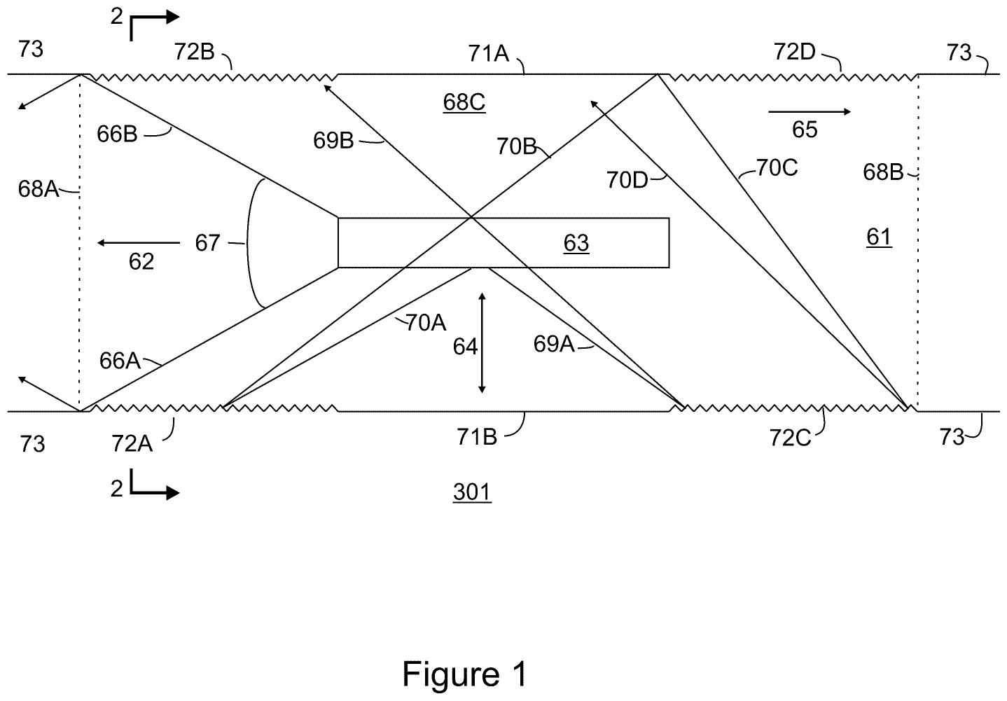

FIG. 1 shows a schematic cross section of a duct 61 with ridge arrays positioned to confine radiation generally indicated at 301. Duct 61 has radiation source 63 positioned along central axis of duct 62. Radiation source 63 may for example be a discharge tube emitting ultraviolet radiation. Smooth flat duct walls 71A and 71B extend the length of radiation source 63 and act to reflect radiation emitted radially back toward radiation source 63 as shown at 64. Ridge arrays 72A and 72B abut flat reflective surfaces 71A and 71B, respectively and extend in the direction of duct axis 62 toward low reflectivity surfaces 73. Duct axis 62 corresponds to the valve direction for ridge arrays 72A and 72B. Similarly, ridge arrays 72C and 72D abut flat reflective surfaces 71A and 71B, respectively and extend in the direction of valve direction 65 toward low reflectivity surfaces 73. Preferably flat surfaces 71A and 71B together with ridge arrays 72A, 72B, 72C, and 72D have reflectivity at a design wavelength of at least 90%. For example aluminum is at least 90% reflective at UV wavelengths. More preferably the reflectivity is at least 95% at the design wavelength. For example, silver and gold are at least 95% reflective at visible and infrared wavelengths, respectively. Most preferably the reflectivity is at least 99% at the design wavelength. For example, the dielectric surfaces disclosed in the above referenced CHAMBER and WALL patents by the current inventors may be at least 90%, 95% or 99% reflective at a design wavelength in the UV, visible or infrared regions. Preferably surfaces 71A and 71B and the ridge faces of ridge array 72A, 72B, 72C, and 72D are flat with RMS deviation from flatness less than ¼ of the design wavelength. More preferably surfaces 71A and 71B and the ridge faces of ridge array 72A, 72B, 72C, and 72D are flat with RMS deviation from flatness less than 1/20 of the design wavelength. Low reflectivity surfaces 73 may for example be steel duct wall with typical reflectivity of about 28%. In some embodiments, low reflectivity surfaces 73 are the base substrate for the ridge arrays 72A, 72B, 72C, 72D and flat reflective surfaces 71A and 71B.

The duct region between the low reflectivity surfaces 73 defined by boundaries 68A and 68B is the radiation zone 68C.

Rays emitted into the angle 67 bounded by rays 66A and 66B travel along duct axis 62 or are incident on low reflectivity surfaces 73 and contribute to the radiation flux between radiation zone boundaries 68A and 68B only once. Rays emitted outside of angle 67 are incident onto the ridge arrays or flat reflective surfaces within the radiation zone and contribute to the radiation flux within the radiation zone a plurality of times.

Ray 69A with amplitude 1.0 is emitted from radiation source 63 and is incident on ridge array 72C with reflectivity 90% and is reflected toward ridge array 72B as ray 69B with amplitude 0.90. That is ray 69B contributes 0.90 energy units to each volume element it passes through (weighted by the path length through the volume element) whereas ray 69A contributes 1.0 energy units to each volume element it passes through.

Ray 70A with amplitude 1.0 is emitted from radiation source 63 and is incident on ridge array 72A with reflectivity 90% and is reflected toward flat reflective region 71A as ray 70B with amplitude 0.90. Ray 70B is reflected by flat region with reflectivity 90% toward ridge array 72C as ray 70C with amplitude 0.81. Ray 70C is incident on ridge array 72C with reflectivity 90% and is reflected toward flat reflective region 71A as ray 70D with amplitude 0.73. Hence the ray amplitude decreases after each reflection. Increasing the reflectivity at the flat surfaces and ridge surfaces increases the contribution of reflected rays to the energy density in the radiation zone 68. The ridge angles are selected to increase the number of reflections between reflective surfaces and hence the radiation energy density within the radiation zone. The optimal ridge angles depend upon the specific geometry of the radiation source and duct walls.

Empirically, optimal ridge angles α are between 35 degrees and 55 degrees. Empirically, optimal ridge angles β are between 45 degrees and 65 degrees.

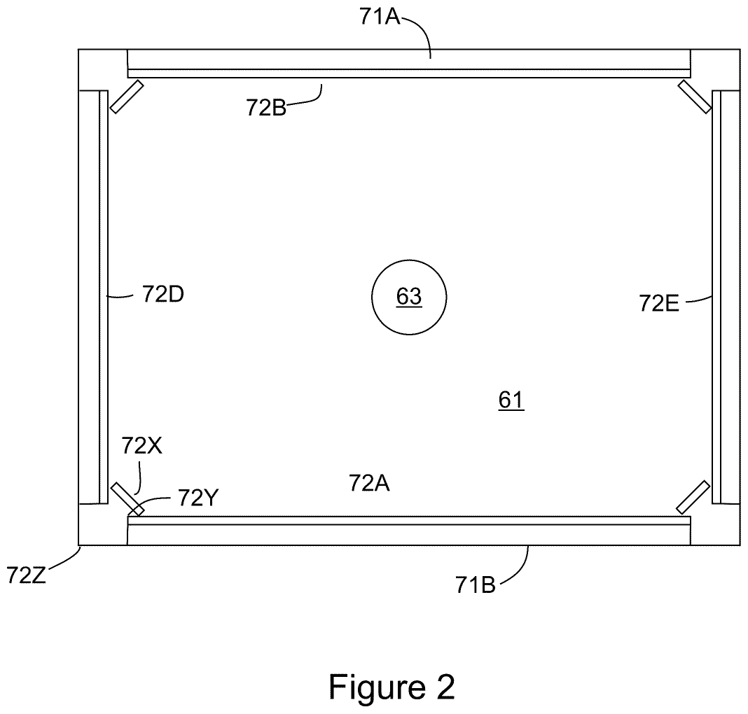

FIG. 2 shows the duct 61 and the source 63 at the center of the duct. It also shows that the reflective ridge arrays 72A and 72B are arranged in opposed positions at the top and bottom walls of the duct. In addition further arrays 72D and 72E are arranged on side walls at right angles to the top and bottom. All are aligned in the duct so that they commence and finish at the same transverse plane. It will be noted that the arrays are located just beyond the end of the source so as to best interact with rays which are transmitted along the duct form the source.

The arrays are formed typically of a film or layer which is applied onto the wall of the duct and this can be applied onto the interior surface of an existing duct. As such ducts often vary slightly in wall width, corner pieces of a reflective material such as aluminum strips and indicated at 72X are inserted at the corners 72Z of the duct to span between edges 72Y of the reflective array to cover the end edges of the array to prevent the light accessing the base material of the array. This is important where the radiation is UV for purposes of sterilization since this can be very corrosive to a supporting plastics material.

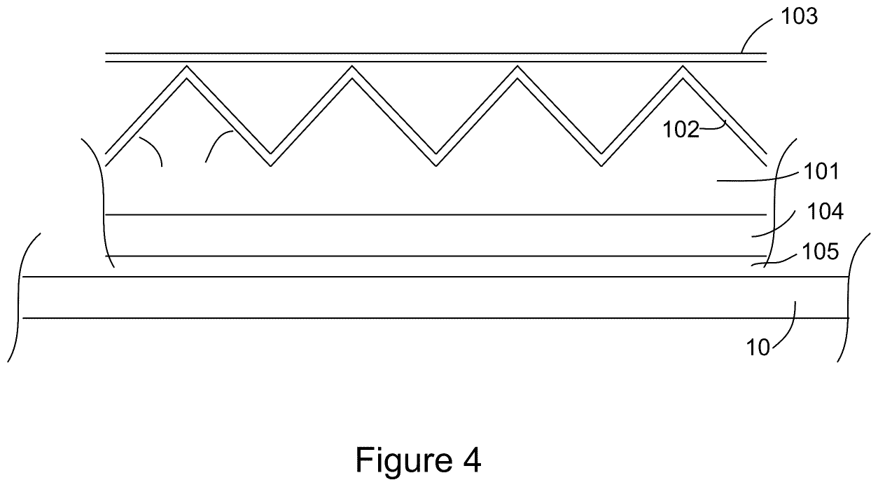

FIG. 4 shows an enlarged cross section of the reflective ridge array. This includes a formed base a material 101 which is shaped to define the required shape of the ridges. On top of the upper surface of the base material 101 is applied the reflective material typically a dielectric mirror layer 102 which is thin so that it follows the upper surface of the recesses in the ridges.

On top of the apexes of the ridges is provided a transparent sheet 103 covering the array of ridges at the apexes thereof. This therefore encloses the recesses between the ridges to prevent the accumulation of dust and the collection of pockets of stationary air or fluid within the duct so as to provide a smooth surface to the fluid flowing in the duct.

The reflective surface includes an insulating layer 104 to reduce transfer of heat through the reflective surface. This can increase the insulation of the duct to prevent heat or cool loss to the environment and also to maintain a more stable temperature within the duct which can be beneficial to the operation of the source which is often heat sensitive. The ridge array can include an adhesive layer 105 by which it can be easily applied onto the interior surface of the duct wall as a simple retro-fit.

A formed array of this structure can be applied to or integral to a body forming a tile with predetermined dimensions. This tile can be easily inserted into an existing duct.

A formed array of this structure can be applied to or integral to a sheet of material that can be cut to size. That is the structure of FIG. 4 can be supplied as a sheet which is then cut to length and width to fit within the duct on the walls of the duct.

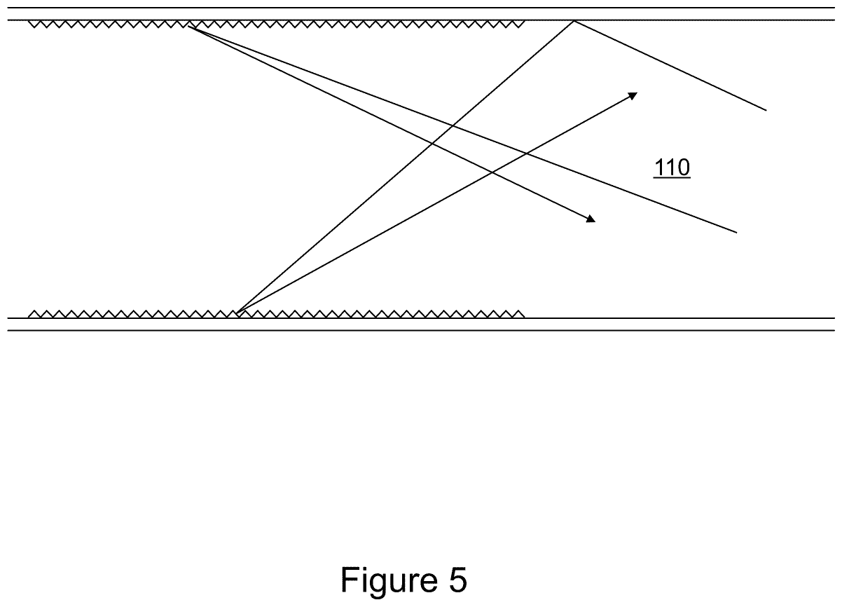

FIG. 5 shows the reflective ridge arrays as described above used in a channel or chamber 110 containing radiation where the first and second reflective ridge surfaces are arranged to increase an amount of the radiation within the chamber by reflecting the radiation back into the duct and to reduce an amount of radiation escaping the chamber at an exit location. This therefore acts as a radiation valve to control escape of the radiation from the chamber and the array of ridges acts to form a restriction to flow of radiation along the volume.

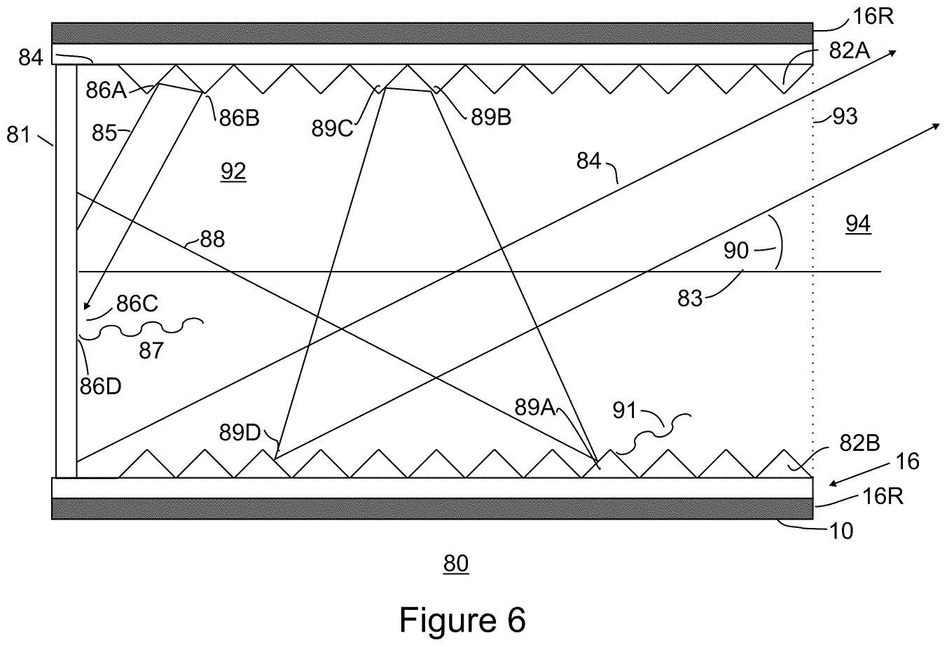

FIG. 6 schematically shows a cross section of a directional thermal radiator generally indicated at 80 with planar thermal radiation source 81 and ridge arrays indicated at 82A and 82B on substrate 16 parallel to normal 83 of thermal radiation source 81. As shown at 16R, substrate 16 may optionally include a thermal insulation layer to reduce conduction from ridge arrays 82A and 82B to base surface 10. Planar thermal radiation source 81 may be approximated in an ideal case as a black body source emitting radiation with a Lambertian angular distribution (cos(θ)) relative to plane normal 83. Ridge arrays 82A and 82B include a short flat segment 84 proximate to thermal radiator 84 which allows rays emitted near the edges of thermal radiator to enter radiator chamber 92 enclosed on three sides by thermal radiation source 81 and ridge arrays 82A and 82B. Radiation chamber 92 extends to the edge of ridge arrays 93, which may be open to free space 94. Optionally radiation chamber 92 and free space may be separated by a transparent window located proximate to edge 93 (not shown). The window functions to prevent air flow between chamber 92 and free space 94 and possible contamination of internal surfaces of chamber 92 with dust. Five types of radiation are emitted and propagate within radiation chamber 92. Firstly, radiation emitted by thermal source 81 may pass through radiation chamber 92 directly into free space 94 without interaction with ridge arrays 82A and 82B as shown at 84. This radiation is limited to an angular range determined by the length of the ridge arrays and the gap between ridge arrays 82A and 82B. That is the radiation transmitted to free space 94 is partially collimated and the degree of collimation can be manipulated by adjusting the ridge array length and the gap between ridge arrays. The degree of collimation may be increased by sub-dividing a radiating area into sub-regions and partitioning each sub-region with ridge array walls. For example, the sub-regions may form a hexagonal grid. Further, the distance from the radiating surface and the ridge arrays may vary, such that the peak of a ridge in a first region corresponds to a valley in the ridge of a second region.

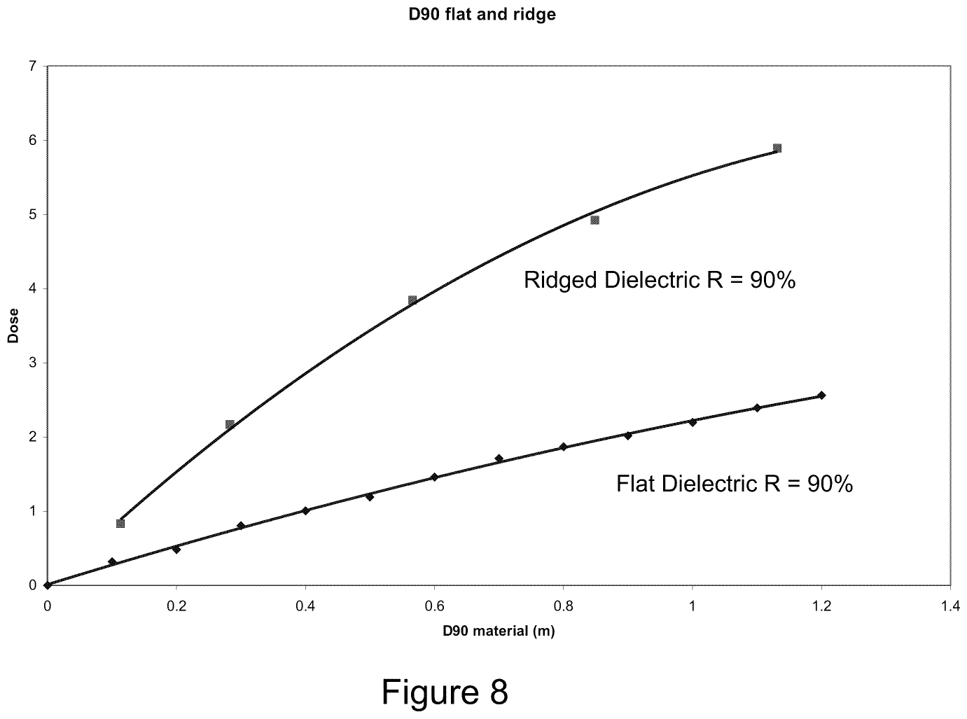

Secondly, a ray 85 emitted by thermal source 81 may be incident on ridge arrays and reflected as shown at 86A and 86B toward a location 86C on thermal source 81. In this case, the photon energy is absorbed by thermal source 81 increasing the temperature proximate to the absorption site, and possibly introducing a thermal gradient within thermal source 81 as the flux of incident photons at a location on the thermal source 81 depends upon where the location is relative to structures of the surrounding ridge arrays 82A and 82B. Ridge arrays 86A and 86B function to increase the operating temperature of thermal source 81 thereby shifting the distribution of emission wavelengths to shorter wavelengths in accordance with the Planck distribution (in the ideal black body case). Those skilled in the art will realize that the wavelength distribution may be altered from the ideal case by choice of materials and geometry (photonic crystal structures, for example). For thermal equilibrium, the thermal energy absorbed is re-emitted. The secondary photons from the thermal source shown at 87 are the third type of radiation in the chamber. The re-emitted secondary photons 87 have the same wavelength and angular distribution as the primary photons 85, but may have a different distribution of origin points, thereby influencing the collimation of the output beam. A substantial fraction of primary photons may be re-absorbed by the thermal source 81. In the simulation shown in FIG. 8 the fraction is 57%.

Fourth, radiation emitted by thermal source 81 may be incident on ridge arrays 82A and 82B multiple times and be partially absorbed at each incidence as shown by ray 88 incident on ridge arrays at 89A, 89B, 89C and 89D before passing into free space 94. The energy absorbed at 89A (about 9% of photons in a numerical simulation) causes local heating of the ridge array and re-emission of secondary photons as shown at 91 (fifth type of radiation in chamber 92). The same process occurs at locations 89B, 89C, and 89D. The temperature of ridge array 82A and 82B may vary by location dependent on the radiation absorbed (generally lower than thermal source 81) resulting in secondary photons with wavelength distribution of the emission location. Secondary emission from the ridges adds longer wavelength photons that are less collimated than photons emitted from thermal source 81.

The first and second reflective ridge surfaces are substantially flat as shown but can be slightly concave so that the radiation incident on the reflective ridge surface is primarily reflected in a predetermined reflection direction.

As shown in FIG. 1, the reflective surface includes two portions which are positioned at first and second positions spaced longitudinally of the volume with the source located between the first and second positions so as to increase an amount of the radiation contained between the two positions.

As described above, the reflectivity of each reflective ridge surface of the incident radiation is generated by a dielectric mirror to provide high reflectivity. This is thus tailored to the wave length of the radiation and also to the predetermined angles of incidence determined by the geometry relative to the source.

The first reflective ridge surface of a first ridge and second reflective ridge surface of a second ridge intersect at the base as shown at surfaces 23 and 32 in FIG. 3. However there may also be a separation of the intersection at the bases as shown at surface 13 forming a region of flat reflective substrate material.

The ridge width of a first ridge 30 is different from the ridge width of a second ridge 40. However they may all be a common width as shown in other figures.

The arrangement herein allows use of a reflective system where the ridges are arranged at a height so as to project from the base to a position which does not interfere with the operation of material in the volume or duct. Thus the reflectors can be easily retrofitted into an existing duct without interfering with the designed operation.

As shown in FIG. 3 the ridges are arranged relative to the source so that first reflective ridge surface 22 reflects back an incident ray as shown at 7A toward the source as shown at 7B and the second reflective ridge surface 23 reflects a ray 8A into a ray 8B incident onto the first reflective ridge surface 32 of the next adjacent ridge 30 from which it is reflected back along the duct as indicated at 8C.

FIG. 8 shows the relationship between total dose and surface length for flat reflective material (bottom curve) and a ridge array (top curve). The dose, integrated for unit time over the length of the radiation zone, increases as the square root of the ridge array length and linearly with the flat reflective material length. The model geometry used for FIG. 8 is shown in FIG. 1 with duct dimensions of a large HVAC duct. The indicated length is the length on both sides of the radiation source, so that the extent of material beyond the radiation source on one side is half of the indicated length. The bottom curve corresponds to flat reflective material extending beyond the radiation source edge on both sides by half of the indicated length. Conceptually this is equivalent to assigning the ridge arrays 72A, 72B, 72C, and 72D set to zero ridge height. The surface length for the ridge array is calculated by adding the lengths of the facet sides (22 and 23 in FIG. 3) for each ridge in the array and for both sides of the radiation source. Hence, for the same amount of reflective material, the ridge array gives more than double the dose for the HVAC duct modeled. Further, the superior performance of the ridge array fits within a linear dimension along the duct axis on the order of 1/sqrt(2) (exact for all ridge angles 45 degrees) of the linear dimension required by a flat reflective surface of the same length.

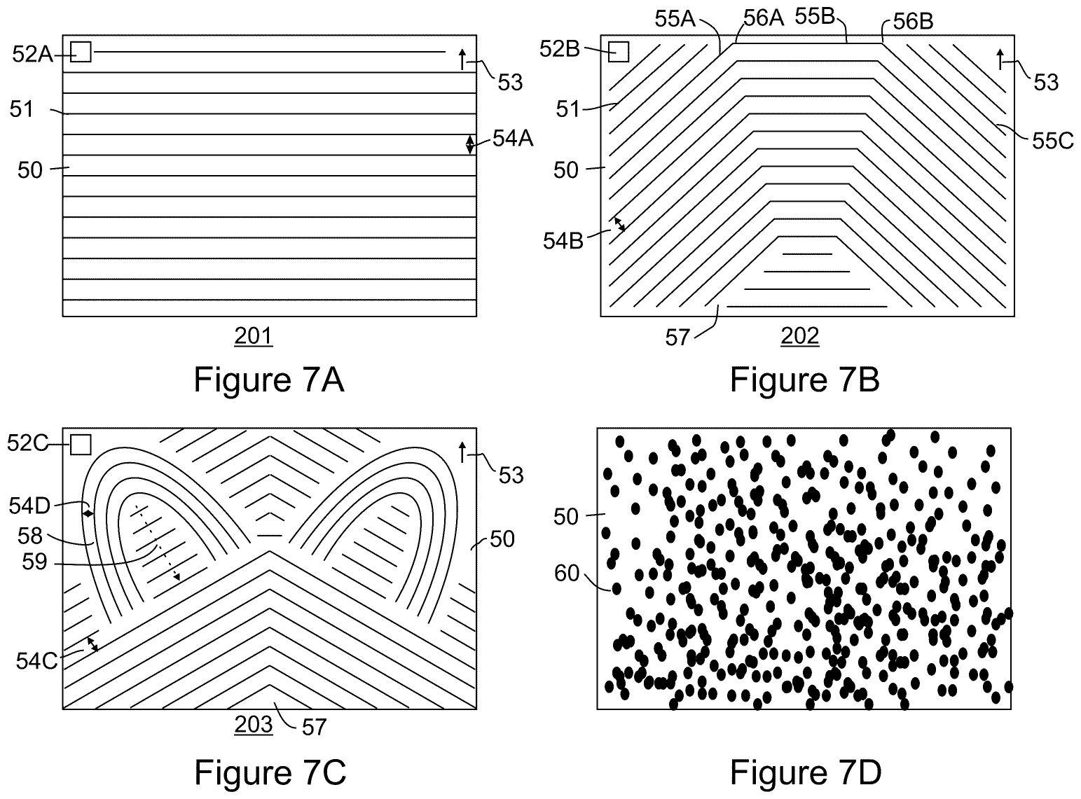

FIGS. 7A, 7B, and 7C schematically show top views of ridge arrays illustrating different features of ridge arrays that may be used individually or in any combination within the scope of the invention. FIG. 7D shows a prior art isotropic scattering arrangement. Like features are labeled with the same numbers. The numeric labels may have a letter appended to indicate the value of the labeled parameter may be different between different embodiments.

FIG. 7A shows a schematic top view of one embodiment of the invention with an array of parallel ridges generally indicated at 201 carried on a substrate material 50. In some embodiments the substrate 50 is a rigid tile with fixed dimensions. Preferably the fixed dimensions are selected such that an integral number of ridge tiles is commensurate with the dimensions of a base substrate. For example, a duct with rectangular cross section 2 units×3 units may be covered with tiles 1 unit wide (2 tiles in the first dimension and 3 tiles in the second direction). Alternately standard tiles may be made with widths of 2 units and 3 units. The length of the tiles (and number of ridges) oriented along the duct axis may be selected based on the application specific optical valve performance desired. In general, increasing the length of ridge material along the duct axis intercepts and redirects a greater fraction of incident radiation. In some embodiments the ridge substrate material 50 is a flexible sheet that may be cut and formed to conform with a base surface. For example the ridge substrate material may be in the form of a roll. The ridge lines shown at 51 correspond to the ridge apex 24 as best seen in FIG. 3. The ridge substrate material 50 may include a region 52A that contains information about at the ridges 51 and the substrate material 50. The information may for example include the dimensions of the material, the ridge angles, height and pitch, the number of ridges and their placement or pattern on the substrate material, the ridge reflectivity and design wavelength. In some embodiments, the information region 52A may include information about a decorative pattern on the ridge array 201 recognizable at visual wavelengths between 400 nm and 700 nm. The information region may include a unique identity code that links to parameter values in an external database. The ridge substrate may include a valve direction indicator as shown at 53. As shown the arrow points in the direction of incident radiation (away from a source of incident radiation). In some embodiments, the opposite convention may be followed in which the arrow points toward the source of incident radiation, in which case the discussion of valve direction herein is to be interpreted in the reversed frame of reference. The ridges 51 have a periodic spacing as indicated at 54A.

FIG. 7B shows a schematic top view of a second embodiment of the invention with an array of ridges 51 generally indicated at 202 carried on a substrate material 50. The substrate material may include an information region 52B with different parameters than 52A. The substrate material may include a valve direction marker 53. The ridge spacing is given at 54B which may be different from 54A. Ridges may consist of a plurality of straight segments 55A, 55B and 55C with a change in ridge direction as indicated at 56A and 56B. The ridge 55A, 55B, 55C as shown is shaped to reflect radiation at increased angle to the valve direction: that is the component of the direction vector perpendicular to the valve direction increases. This arrangement may be advantageous for confining radiation flux to a volume between two or more reflectors. As shown at 57, there may be a flat region between ridge lines.

FIG. 7C shows a schematic top view of a third embodiment of the invention with an array of ridges 51 generally indicated at 203 carried on a substrate material 50. The substrate material may include an information region 52C with different parameters than 52A and 52B. The substrate material may include a valve direction marker 53. Different ridge periods are given at 54C and 54D, which may be different from 54A and 54B. Ridges may be curved to focus radiation in two dimensions as shown at 58. The concentric generally parabolic ridges along axis 59 will each have a different focal length, so the effect of a nested series of parabolic ridges is to focus radiation along a line in the direction of 59 corresponding to the different focal lengths. This feature may be used for example to increase the homogeneity of radiation flux in a reaction volume by directing extra flux to regions of otherwise low flux density. That is the focal length and axis 59 of the parabolic (or curved) features are designed to reflect radiation to a selected low flux density region. As shown at 57, there may be a flat region between ridge lines.

FIG. 7D schematically shows a prior art arrangement of random scattering centers 60 on a substrate 50. In this arrangement all directions of scattering are equally probable, so that on average 50% of the radiation incident on the substrate 50 is scattered in the valve direction away from the source of radiation. While the scattering centers 50 may have very high reflectivity and minimal absorption losses, the 50% of radiation scattered beyond the extent of the substrate may be incident on absorbing surfaces and lost.

FIG. 8 shows the relationship between total dose and surface length for flat reflective material (bottom curve) and a ridge array (top curve). The dose, integrated for unit time over the length of the radiation zone, increases as the square root of the ridge array length and linearly with the flat reflective material length. The model geometry used for FIG. 8 is shown in FIG. 1 with duct dimensions of a large HVAC duct. The indicated length is the length on both sides of the radiation source, so that the extent of material beyond the radiation source on one side is half of the indicated length. The bottom curve corresponds to flat reflective material extending beyond the radiation source edge on both sides by half of the indicated length. Conceptually this is equivalent to assigning the ridge arrays 72A, 72B, 72C, and 72D set to zero ridge height. The surface length for the ridge array is calculated by adding the lengths of the facet sides (22 and 23 in FIG. 3) for each ridge in the array and for both sides of the radiation source. Hence, for the same amount of reflective material, the ridge array gives more than double the dose for the HVAC duct modeled. Further, the superior performance of the ridge array fits within a linear dimension along the duct axis on the order of 1/sqrt(2) (exact for all ridge angles 45 degrees) of the linear dimension required by a flat reflective surface of the same length.

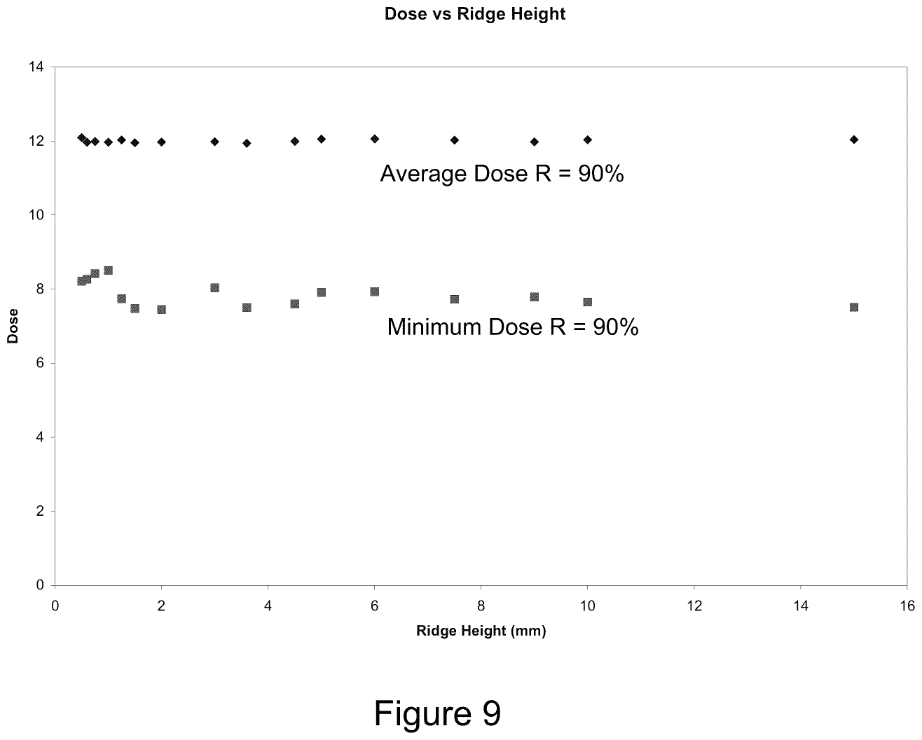

FIG. 9 shows the relationship between dose and ridge height for the geometry shown in FIG. 1. Both the average dose and minimum dose are given. The surprising and unexpected result of modeling by the inventors is that the dose is independent of the ridge scale. This means that the ridges can be made as small as manufacturing methods permit without loss of performance, for example with ridge heights of 0.5 mm to 1.0 mm. In the context of a HVAC duct, this means that the ridge array has minimal impact on air flow.

The ratio of minimum dose to average dose is a measure of homogeneity of the radiation field in the radiation zone. The ratio for the ridge geometry typically falls in the range of 0.6 to 0.7 over a range of duct geometries. If the ridge array is replaced with isotropic reflective material, the homogeneity ratio falls in the same range, but the overall dose is lower for equivalent reflectivity.

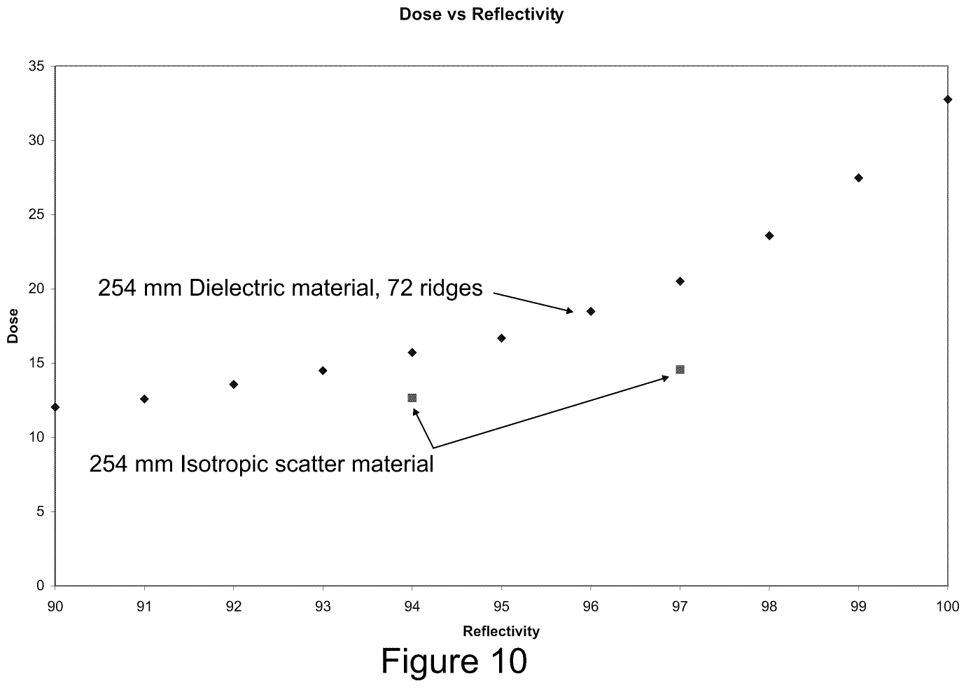

FIG. 10 shows the dependence of dose on the reflectivity of ridge material for 72 ridges spanning 254 mm either side of a radiation source as shown in FIG. 1. For comparison, an equal span of isotropic scattering media with the same reflectivity is shown. The ridge arrangement of the invention gives a 24% higher dose for material with 94% reflectivity and a 41% higher dose for material with 97% reflectivity. That is the advantage conferred by the ridge array arrangement over conventional isotropic scattering media increases with increasing reflectivity.

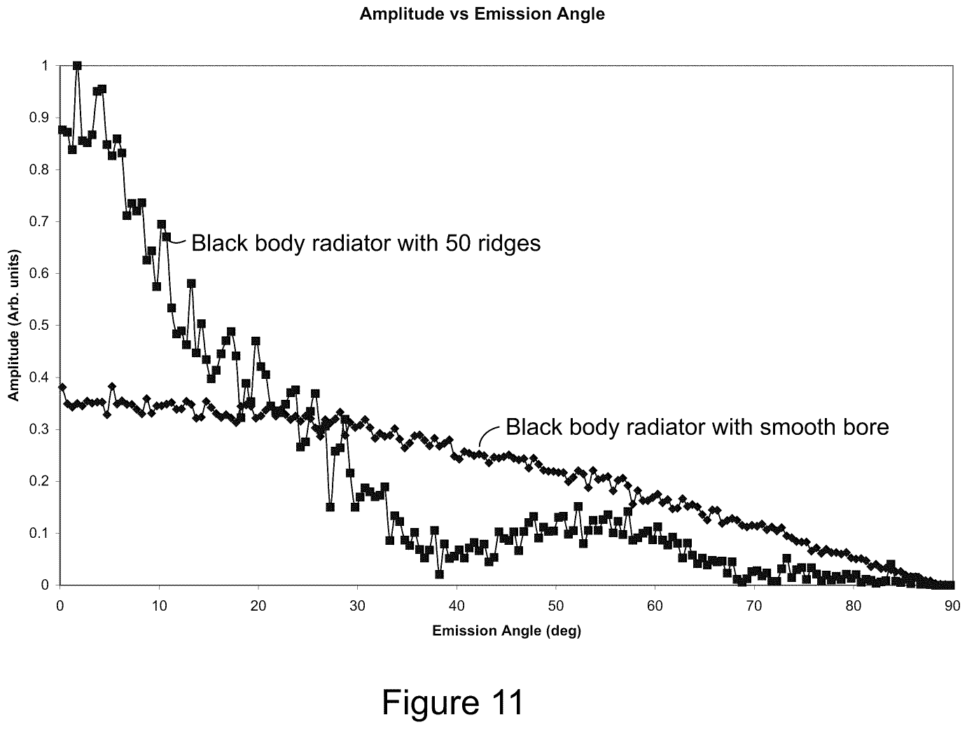

FIG. 11 shows the angular distribution of radiation emitted from the arrangement shown in FIG. 6 into free space 94 as calculated with a numerical simulation tracing 10 million rays emitted from random locations of thermal radiator 81, modeled as a perfect black body radiator with Lambertian angular distribution. The radiator 81 is 100 mm×100 mm and the gap between ridge arrays 82A and 82B is 100 mm. The flat region 84 is 10 mm, and each ridge array contains 50 ridges with ridge height 3 mm for overall length 310 mm. The ridge arrays are modeled as thermally isolated (no conductive loss) perfectly smooth dielectric reflectors with angle and polarization dependent reflectivity. For comparison, the angular distribution of radiation into free space 94 is shown for a smooth bore arrangement with the same overall dimensions, except that the ridge height is zero: that is the surface 84 replaces the ridge arrays 82A and 82B over the length of the ridge arrays. The total radiation emitted into free space 94 is the same for both models. The reflective surfaces of the smooth bore model substantially replicate the Lambertian angular distribution of the black body radiator, aside from minor attenuation at large angles due to absorption. The ridge array model collimates the output with about 2.5× more amplitude at angles less than 5 degrees and lower amplitude at angles greater than 25 degrees. The ridge array model further shows local minima and maxima that is dependent upon the ridge array periodicity. These features may be smeared out, or enhanced, by altering the shape and period of the ridges.

1. A method for regulating flow of electromagnetic radiation comprising
providing a source of electromagnetic radiation;
arranging the source to emit electromagnetic radiation in a direction into a volume,
placing a reflective surface in the volume to receive the radiation thereon;
defining on the reflective surface an array of two or more ridges which are arranged side by side in a first direction across the reflective surface;
wherein each ridge projects forwardly from a base toward the source;
wherein each ridge has a first reflective ridge surface and a second reflective ridge surface where the first and second surfaces extend longitudinally in a second direction transverse to the first direction;
wherein the first reflective ridge surface and the second reflective ridge surface converge toward an apex extending longitudinally along the second direction;
wherein the first reflective ridge surface and the second reflective ridge surface are arranged so that at least some of the radiation impinges on at least one of the first reflective ridge surface and the second reflective ridge surface;
wherein the first reflective ridge surface and the second reflective ridge surface are arranged such that the radiation incident on the first reflective ridge surface is primarily reflected in a first reflection direction and such that the radiation incident on the second reflective ridge surface is primarily reflected in a second reflection direction different from that of the first reflection direction.

2. The method according to claim 1 wherein the volume comprises a duct section and the source is arranged for sterilizing air in the duct section wherein the first and second reflective ridge surfaces are arranged to increase an amount of the radiation within the duct section by reflecting the radiation back into the duct section and to reduce an amount of radiation escaping the duct section.

3. The method according to claim 1 for restricting flow of radiation from a source of electromagnetic radiation in a direction from an upstream location to a downstream location where first and second of the ridges are arranged relative to the source so that first reflective ridge surface reflects primarily toward the upstream location and second reflective ridge surface reflects onto the first reflective ridge surface of a next adjacent ridge and thereby primarily toward the upstream location so that the array of ridges acts to form a restriction to flow of radiation toward the downstream location.

4. The method according to claim 1 for directing radiation from a source in a required direction where the source of electromagnetic radiation acts to direct radiation partly in the required direction, the method comprising:
providing a reflective shroud at the source for guiding the radiation emitted from the source which is not in the required direction;
the reflective shroud comprising:
one or more reflective surfaces at least partly surrounding the source at locations different from the required direction and arranged to receive at least part of the radiation incident thereon;
the or each reflective surface being arranged such that a portion on one side of the source generally opposes a portion on an opposite side;
the first and second reflective ridge surfaces being arranged such that the radiation incident on the first and second reflective ridge surfaces of each portion is reflected toward the source, toward the opposed portion, or toward the required direction;
wherein radiation directed toward the source is absorbed and re-emitted.

5. The method according to claim 1 wherein the first and second reflective ridge surfaces are substantially flat or slightly concave so that the radiation incident on the reflective ridge surface is primarily reflected in a predetermined reflection direction.

6. The method according to claim 1 wherein the reflective surface includes two portions which are positioned at first and second positions spaced longitudinally of the volume with the source located between the first and second positions so as to increase an amount of the radiation contained between the two positions.

7. The method according to claim 1 wherein the reflectivity of each reflective ridge surface of the incident radiation is at least 80% preferably at least 90% and most preferably at least 95% and wherein the reflectivity of each reflective ridge surface of the incident radiation is generated by a dielectric mirror.

8. The method according to claim 1 wherein the first reflective ridge surface and the second reflective ridge surface converge to a common apex at a line.

9. The method according to claim 1 wherein the first reflective ridge surface and second reflective ridge surface are joined at the apex by an intermediate reflective ridge surface.

10. The method according to claim 1 wherein the first reflective ridge surface of a first ridge and second reflective ridge surface of a second ridge intersect at the base.

11. The method according to claim 1 wherein the first reflective ridge surface of a first ridge and second reflective ridge surface of a second ridge are separated at the base by a region of flat reflective substrate material.

12. The method according to claim 1 wherein the ridge width of a first ridge is different from the ridge width of a second ridge.

13. The method according to claim 1 the first reflective ridge surface and second reflective ridge surface are formed by a layer of a reflective substrate material which is applied onto a base support shaped to define the ridges.

14. The method according to claim 13 wherein the reflective substrate material covers the ridges and extends onto a base surface.

15. The method according to claim 14 wherein the substrate material includes a selected direction mark.

16. The method according to claim 1 wherein the reflective substrate surface is integral with one of the following:
a tile with predetermined dimensions;
a sheet of material that can be cut to size;
the surface of an architectural structure;
a decorative material.

17. The method according to claim according to claim 1 wherein two or more ridge arrays with different selected directions are mounted on a base surface at different sides of a radiation source.

18. The method according to claim 1 wherein the ridge height is less than 10 mm and preferably less than 0.5 mm.

19. The method according to claim 1 wherein there is provided a transparent sheet covering the array of ridges at the apexes thereof.

20. The method according to claim 1 wherein the reflective surface includes an insulating layer to reduce transfer of heat through the reflective surface.

21. The method according to claim 1 wherein an angle of first reflective ridge surface to the base surface is different from the angle of the second reflective ridge surface to the base surface.

22. The method according to claim 1 wherein an angle of first and second reflective ridge surfaces to the base surface lie in the range 35 to 55 degrees.

23. The method according to claim 1 wherein a sum of the angle of first reflective ridge surface to the base surface and the angle of the second reflective ridge surface to the base surface is different from 90 degrees.

24. The method according to claim 1 wherein there are walls surrounding a volume where a portion of the reflective surface is located at or adjacent each wall and the portions are aligned along the volume.

25. The invention according to claim 1 wherein the reflectivity of the ridge array at a design wavelength may change in response to an external stimulus wherein the external stimulus is a temperature or a voltage applied to the substrate layer.

26. The invention according to claim 1 wherein at least one ridge geometric parameter may be modified in response to an external stimulus wherein the external stimulus is one of electrical, mechanical, or thermal.

27. The invention according to claim 1 wherein the substrate material includes pores which allow the passage of molecules through the substrate material.
